(19) United States
(12) Patent Application Publication
(10) Pub. No.: US 2022/0047489 A1
(43) Pub. Date: Feb. 17, 2022

(54) COSMETIC PRODUCT FOR TREATING A KERATIN MATERIAL HAVING ANTI-POLLUTION EFFECTS

(71) Applicant: Henkel AG & Co. KGaA, Duesseldorf (DE)

(72) Inventors: RENE KROHN, Norderstedt (DE); ERIK SCHULZE ZUR WIESCHE, Bielefeld (DE); TORSTEN LECHNER, Langenfeld (DE)

(73) Assignee: Henkel AG & Co. KGaA, Duesseldorf (DE)

(21) Appl. No.: 17/290,207

(22) PCT Filed: Oct. 31, 2019
(86) PCT No.: PCT/EP2019/079765

§ 371 Date: Apr. 29, 2021

(30) Foreign Application Priority Data
Oct. 31, 2018 (DE) 10 2018 218 647.2

(51) Int. Cl. A61K 8/60; A61K 8/58; A61Q 5/00

(52) U.S. Cl. CPC A61K 8/602; A61Q 5/00; A61K 8/585

(57) ABSTRACT

The application discloses a cosmetic agent for treating a keratinous material comprising a) at least one organic silicon compound containing one to three silicon atoms, and b) rutin sulfate.

### CROSS-REFERENCE TO RELATED APPLICATION

This application is a U.S. National-Stage entry under 35 U.S.C. § 371 based on International Application No. PCT/EP2019/079765, filed Oct. 31, 2019, which was published under PCT Article 21(2) and which claims priority to German Application No. 102018218647.2, filed Oct. 31, 2018, which are all hereby incorporated in their entirety by reference.

### TECHNICAL FIELD

The application concerns cosmetic agents for treating a keratinous material with rutin sulfate and their use.

### BACKGROUND

Air and water contaminants can have a detrimental effect on skin and hair. The most important air pollutants include polycyclic aromatic hydrocarbons, volatile organic compounds, nitrogen oxides (NOx), particles, and cigarette smoke. The action of various atmospheric pollutants can be intensified in the presence of other atmospheric pollutants and under UV radiation action.

It is known that the toxicity of gaseous pollutants of air, such as sulfur dioxide, ozone, and nitrogen oxides, is related in particular to their initiator activity for free radicals which cause damage to living organisms. Free radicals are metabolites that also occur naturally in the body. In large quantities, free radicals can promote irritation and inflammation and accelerate the process of aging. In this case, one speaks of “oxidative damage” Free radicals can also cause visible hair damage, for example, as a reduction in shine as well as grip and/or fading of hair color.

Particulate matter is a complex mixture containing metals, minerals, organic toxins, and/or biological materials. They can also promote the formation of free radicals.

It is known that antioxidant compounds can be used as free radical scavengers, mitigating the effects of free radical formation. Bioflavonoids such as rutin, catechin and naringin, for example, have the ability to “catch” free radicals.

WO 2018/115059 A1 describes organosilicon compounds from the group of silanes comprising at least one hydroxy group and/or hydrolyzable group. Due to the presence of hydroxy groups and/or hydrolyzable groups, silanes are reactive substances that hydrolyze or oligomerize or polymerize in the presence of water. The oligomerization or polymerization of the silanes, initiated by the presence of the water, ultimately leads to the formation of a film when applied to a keratinous material.

### BRIEF SUMMARY

In an exemplary embodiment, a cosmetic agent is provided for the treatment of a keratinous material. An exemplary cosmetic agent comprises at least one organic silicon compound comprising one to three silicon atoms, and Rutin sulfate.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

There is a need for a product that can reduce or prevent the harmful effects of air and water contaminants on a keratinous material, especially hair (“anti-pollution” effect). In particular, there is a need for a product that reduces or prevents the formation of free radicals by air and water contaminants and/or renders formed free radicals harmless.

This problem is solved by a cosmetic agent for the treatment of a keratinous material, comprising

a) at least one organic silicon compound containing one to three silicon atoms, and
b) Rutin sulfate.

Keratinous material includes hair, skin, nails (such as fingernails and/or toenails). Wool, furs and feathers also fall under the definition of keratinous material.

Preferably, keratinous material is understood to be human hair, human skin and human nails, especially fingernails and toenails. In particular, keratinous material is understood to mean human hair, especially head and/or beard hair.

As a first ingredient, the agent for treating a keratinous material contains at least one organic silicon compound containing one to three silicon atoms. Preferred organic silicon compounds are selected from silanes having one, two, or three silicon atoms, wherein the organic silicon compound comprises one or more hydroxyl groups and/or hydrolyzable groups per molecule.

Organic silicon compounds, alternatively called organosilicon compounds, are compounds which either have a direct silicon-carbon bond (Si—C) or in which the carbon is bonded to the silicon atom via an oxygen, nitrogen or sulfur atom. The organic silicon compounds of interest herein are compounds containing one to three silicon atoms. Exemplary organic silicon compounds for use herein preferably contain one or two silicon atoms.

According to IUPAC rules, the term silane stands for a group of chemical compounds based on a silicon skeleton and hydrogen. In organic silanes, the hydrogen atoms are completely or partially replaced by organic groups such as (substituted) alkyl groups and/or alkoxy groups. In organic silanes, some of the hydrogen atoms may also be replaced by hydroxy groups.

The agent for treating a keratinous material contains at least one organic silicon compound, preferably selected from silanes having one, two, or three silicon atoms, wherein the organic silicon compound comprises one or more hydroxyl groups or hydrolyzable groups per molecule.

In a particularly preferred embodiment, the agent for treating a keratinous material comprises at least one organic silicon compound selected from silanes having one, two, or three silicon atoms, the organic silicon compound further comprising one or more basic groups and one or more hydroxyl groups or hydrolyzable groups per molecule.

This basic group can be, for example, an amino group, an alkylamino group, or a dialkylamino group, which is preferably connected to a silicon atom via a linker. The basic group is preferably an amino group, a C₁-C₆ alkylamino group, or a di(C₁-C₆)alkylamino group.

The hydrolyzable group(s) is (are) preferably a C₁-C₆ alkoxy group, especially an ethoxy group or a methoxy group. It is preferred that the hydrolyzable group be directly bonded to the silicon atom. For example, if the hydrolyzable group is an ethoxy group, the organic silicon compound preferably contains a structural unit R′R″R′″Si—O—CH₂—CH₃. The residues R′, R″ and R′″ represent the three remaining free valences of the silicon atom.

Particularly good results were obtained when the agent for treating a keratinous material contains at least one organic silicon compound of formula (I) and/or (II).

The compounds of formulae (I) and (II) are organic silicon compounds selected from silanes having one, two, or three silicon atoms, the organic silicon compound comprising one or more hydroxyl groups and/or hydrolysable groups per molecule.

In another very particularly preferred embodiment, the agent for treating a keratinous material comprises at least one organic silicon compound of formula (I) and/or (II),

R₁R₂N-L-Si(OR₃)ₐ(R₄)_{b} (I),

where
R₁, R₂ independently represent a hydrogen atom or a C₁-C₆ alkyl group,
L is a linear or branched C1-C20 divalent alkylene group,
R₃ represents a hydrogen atom or a C₁-C₆ alkyl group,
R₄ represents a C₁-C₆ alkyl group,
a, stands for an integer from 1 to 3, and
b stands for the integer 3-a,

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),

where
R5, R5′, R5″ independently represent a hydrogen atom or a C₁-C₆ alkyl group,
R6, R6′ and R6″ independently represent a C₁-C₆ alkyl group,
A, A₁, A₂, A₃ and A₄ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

(A″″)-Si(R₆″)_{d}″(OR₅″)ₑ″ (III),

where
c, stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1, and
h stands for 0 or 1,
provided that at least one of e, f, g, and h is different from 0.

The substituents R₁, R₂, R₃, R₄, R₅, R₅′, R₅″, R₆, R₆′, R₆″, R₇, R₈, L, A′, A″″ and A″″ in the compounds of formula (I) and (II) are explained below as examples:

Examples of a C₁-C₆ alkyl group are the groups methyl, ethyl, propyl, isopropyl, n-butyl, s-butyl and t-butyl, n-pentyl and n-hexyl. Propyl, ethyl and methyl are preferred alkyl radicals. Examples of a C₂-C₆ alkenyl group are vinyl, allyl, but-2-enyl, but-3-enyl and isobutenyl, preferred C₂-C₆ alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino C₁-C₆ alkyl group are the aminomethyl group, the 2-aminoethyl group, the 3-aminopropyl group. The 2-aminoethyl group is particularly preferred. Examples of a linear divalent C₁-C₂₀ alkylene group include the methylene group (—CH₂),), the ethylene group (—CH₂—CH₂—), the propylene group (—CH₂—CH₂—CH₂—) and the butylene group (—CH₂—CH₂—CH₂—). The propylene group (—CH₂—CH₂—CH₂—) is particularly preferred. From a chain length of 3 C atoms, divalent alkylene groups can also be branched. Examples of branched divalent C₃-C₂₀ alkylene groups are (—CH₂—CH(CH₃)—) and (—CH₂—CH(CH₃)—CH₂—).

In the organic silicon compounds of the formula (I)

R₁R₂N-L-Si(OR₃)ₐ(R₄)_{b} (I),

the radicals R₁ and R₂ independently of one another represent a hydrogen atom or a C₁-C₆ alkyl group. In particular, the radicals R₁ and R₂ both represent a hydrogen atom.

In the middle part of the organic silicon compound is the structural unit or the linker -L-, which stands for a linear or branched, divalent C₁-C₂₀ alkylene group.

Preferably, -L- represents a linear, divalent C₁-C₂₀ alkylene group. More preferably -L-stands for a linear divalent C₁-C₆ alkylene group. Particularly preferred -L stands for a methylene group (CH₂—), an ethylene group (—CH₂—CH₂—), propylene group (—CH₂—CH₂—CH₂—) or butylene (—CH₂—CH₂—CH₂—CH₂—). In particular, L stands for a propylene group (—CH₂—CH₂—CH₂—)

The organic silicon compounds of formula (I)

R₁R₂N-L-Si(OR₃)ₐ(R₄)_{b} (I),

one end of each carries the silicon-containing group —Si(OR₃)ₐ(R₄)_{b}

In the terminal structural unit —Si(OR₃)ₐ(R₄)_{b}, R₃ is hydrogen or C₁-C₆ alkyl group, and R₄ is C₁-C₆ alkyl group. In particular, R₃ and R₃ independently of each other represent a methyl group or an ethyl group.

Here a stands for an integer from 1 to 3, and b stands for the integer 3-a. If a stands for the number 3, then b is equal to 0. If a stands for the number 2, then b is equal to 1. If a stands for the number 1, then b is equal to 2.

The best protection against the negative effects of water and/or air pollution (“anti-pollution” effect) could be obtained if the agent for treating a keratinous material contains at least one organic silicon compound of formula (I) in which the radicals R₃, R₄ independently represent a methyl group or an ethyl group.

Furthermore, the best “anti-pollution” effect could be obtained when the agent for treating a keratinous material contains at least one organic silicon compound of formula (I) in which the radical a represents the number 3. In this case the rest b stands for the number 0.

In another preferred embodiment, an agent for treating a keratinous material comprises at least one organic silicon compound of formula (I),

where
R₃, R₄ independently of one another represent a methyl group or an ethyl group,
a stands for the number 3, and
b stands for the number 0.

In another preferred embodiment, the agent for treating a keratinous material comprises at least one organic silicon compound of formula (I)

R₁R₂N-L-Si(OR₃)ₐ(R₄)_{b} (I),

where
R₁, R₂ both represent a hydrogen atom, and
L represents a linear, divalent C₁-C₆-alkylene group, preferably a propylene group (—CH₂—CH₂—CH₂—) or an ethylene group (—CH₂—CH₂—),
R₃ represents a hydrogen atom, an ethyl group or a methyl group,
R₄ represents a methyl group or an ethyl group,
a stands for the number 3, and
b stands for the number 0.

Particularly well-suited organic silicon compounds of formula (I) are

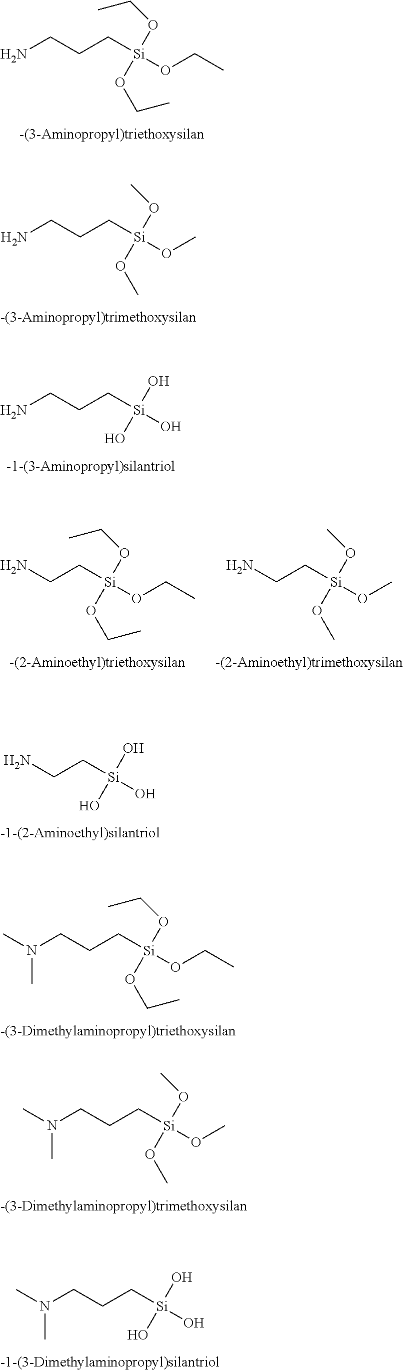

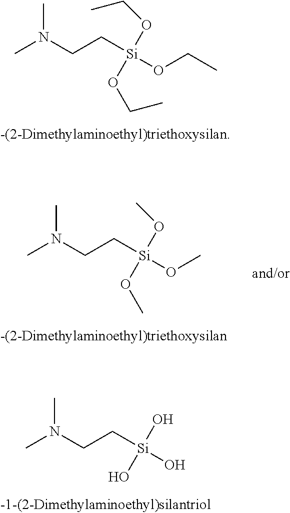

The aforementioned organic silicon compounds of formula (I) are commercially available. (3-aminopropyl)trimethoxysilane, for example, can be purchased from Sigma-Aldrich. (3-Aminopropyl)triethoxysilane is also commercially available from Sigma-Aldrich.

In another embodiment, the composition for treating a keratinous material comprises at least one organic silicon compound of formula (II)

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),

The organosilicon compounds of formula (II) as contemplated herein each carry the silicon-containing groups (R₅O)ₑ(R₆)_{d}Si— and —Si(R₆′)_{d′}(OR₅′)ₑ at both ends.

In the central part of the molecule of formula (II) there are the groups -(A)ₑ- and —[NR₇-(A′)]_{f}- and —[O-(A″)]_{g}- and —[NR₈-(A′″)]ₕ-. Here, each of the radicals e, f, g and h can independently of one another stand for the number 0 or 1, with the proviso that at least one of the radicals e, f, g and h is different from 0. In other words, an organic silicon compound of formula (II) as contemplated herein contains at least one grouping from the group including -(A)- and —[NR₇-(A′)]- and —[O-(A″)]- and —[NR₈-(A′″)]-.

In the two terminal structural units (R₅O)ₑ(R₆)_{d}Si— and —Si(R₆′)_{d′}(OR₅′)ₑ, the radicals R5, R5′, R5″ independently of one another represent a hydrogen atom or a C₁-C₆ alkyl group. The radicals R6, R6′ and R6″ independently represent a C₁-C₆ alkyl group.

Here a stands for an integer from 1 to 3, and d stands for the integer 3-c. If c stands for the number 3, then d is equal to 0. If c stands for the number 2, then d is equal to 1. If c stands for the number 1, then d is equal to 2.

Analogously c′ stands for a whole number from 1 to 3, and d′ stands for the whole number 3-c′. If c′ stands for the number 3, then d′ is 0. If c′ stands for the number 2, then d′ is 1. If c′ stands for the number 1, then d′ is 2.

A very high anti-pollution effect of the agent for the treatment of a keratin material could be obtained if the residues c and c′ both represent the number 3. In this case d and d′ both stand for the number 0.

In a further preferred embodiment, the agent for treating a keratin material contains at least one organic silicon compound of formula (II)

(R₅O)ₑ(R₆)_{d}Si-(A)ₑ-[NR₇-(A′)]_{f}—[O-(A″)]_{g}—[NR₈-(A′″)]ₕ—Si(R₆′)_{d′}(OR₅′)_{e′} (II),

where
R5 and R5′ independently represent a methyl group or an ethyl group,
c and c′ both stand for the number 3, and
d and d′ both stand for the number 0.

If c and c′ are both the number 3 and d and d′ are both the number 0, the exemplary organic silicon compound corresponds to formula (IIa)

(R₅O)₃Si-(A)ₑ-[NR₇-(A′)]_{f}—[O-(A″)]_{g}—[NR₈-(A′″)]ₕ—Si(OR₅′)₃ (IIa).

The radicals e, f, g and h can independently stand for the number 0 or 1, whereby at least one radical from e, f, g and h is different from zero. The abbreviations e, f, g and h thus define which of the groupings -(A)e- and -[NR7-(A′)]f- and —[O-(A″)]g- and —[NR8-(A′″)]h- are located in the middle part of the organic silicon compound of formula (II).

In this context, the presence of certain groups has proved to be particularly advantageous in terms of increasing the “anti-pollution” effect. Particularly good results were obtained when at least two of the residues e, f, g and h stand for the number 1. Especially preferred e and f both stand for the number 1. Furthermore, g and h both stand for the number 0.

If e and f both stand for the number 1 and g and h both stand for the number 0, the organic silicon compound as contemplated herein corresponds to formula (IIb)

(R₅O)ₑ(R₆)_{d}Si-(A)-[NR₇-(A′)]—Si(R₆′)_{d′}(OR₅′)_{e′} (IIb).

The radicals A, A′, A″, A′″ and A″″ independently represent a linear or branched divalent C₁-C₂₀ alkylene group. Preferably the radicals A, A′, A″, A′″ and A″″ independently of one another represent a linear, divalent C₁-C₂₀ alkylene group. Further preferably the radicals A, A′, A″, A′″ and A″″ independently represent a linear divalent C₁-C₆ alkylene group. In particular, the radicals A, A′, A″, A′″ and A″″ independently of one another represent a methylene group (—CH₂—), an ethylene group (—CH₂—CH₂—), a propylene group (—CH₂—CH₂—CH₂—) or a butylene group (—CH₂—CH₂—CH₂—CH₂—). In particular, the residues A, A′, A″, A′″ and A″″ stand for a propylene group (—CH₂—CH₂—CH₂—).

When the radical f represents the number 1, the organic silicon compound of formula (II) contains a structural grouping -[NR7-(A′)]-.

When the radical h represents the number 1, the organic silicon compound of formula (II) contains a structural grouping -[NR8-(A″)]-.

Wherein R₇ and R₈ independently represent a hydrogen atom, a C₁-C₆ alkyl group, a hydroxy-C₁-C₆ alkyl group, a C₂-C₆ alkenyl group, an amino-C₁-C₆ alkyl group or a group of the formula (III)

(A″″)-Si(R₆″)_{d}″(OR₅″)ₑ″ (III),

Very preferably, R₇ and R₈ independently represent a hydrogen atom, a methyl group, a 2-hydroxyethyl group, a 2-alkenyl group, a 2-aminoethyl group or a group of formula (III).

If the radical f represents the number 1 and the radical h represents the number 0, the organic silicon compound as contemplated herein contains the grouping [NR₇-(A′)] but not the grouping —[NR₈-(A′″)]. If the residue R₇ is a grouping of formula (III), the agent for treating a keratin material contains an organic silicon compound with 3 reactive silane groups.

In a further preferred embodiment, the agent for treating a keratin material contains at least one organic silicon compound of formula (II)

(R₅O)ₑ(R₆)_{d}Si-(A)ₑ-[NR₇-(A′)]_{f}—[O-(A″)]_{g}—[NR₈-(A′″)]ₕ—Si(R₆′)_{d′}(OR₅′)_{e′} (II),

where
e and f both stand for the number 1,
g and h both stand for the number 0,
A and A′ independently of one another represent a linear, divalent C₁-C₆ alkylene group, and
R₇ represents a hydrogen atom, a methyl group, a 2-hydroxyethyl group, a 2-alkenyl group, a 2-aminoethyl group, or a group of formula (III).

In another preferred embodiment, the agent for treating a keratin material contains at least one organic silicon compound of formula (II), wherein
e and f both stand for the number 1,
g and h both stand for the number 0,
A and A′ independently of one another represent a methylene group (—CH₂—), an ethylene group (—CH₂—CH₂—), or a propylene group (—CH₂—CH₂—CH₂),
   and
R₇ represents a hydrogen atom, a methyl group, a 2-hydroxyethyl group, a 2-alkenyl group, a 2-aminoethyl group, or a group of formula (III).

Organic silicon compounds of formula (II) that are suitable for solving the problem are

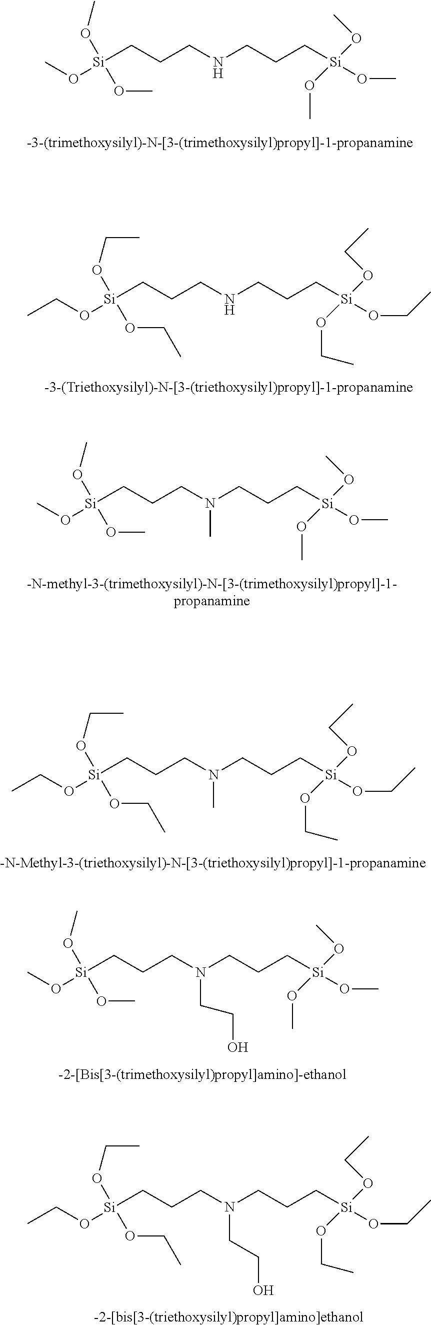

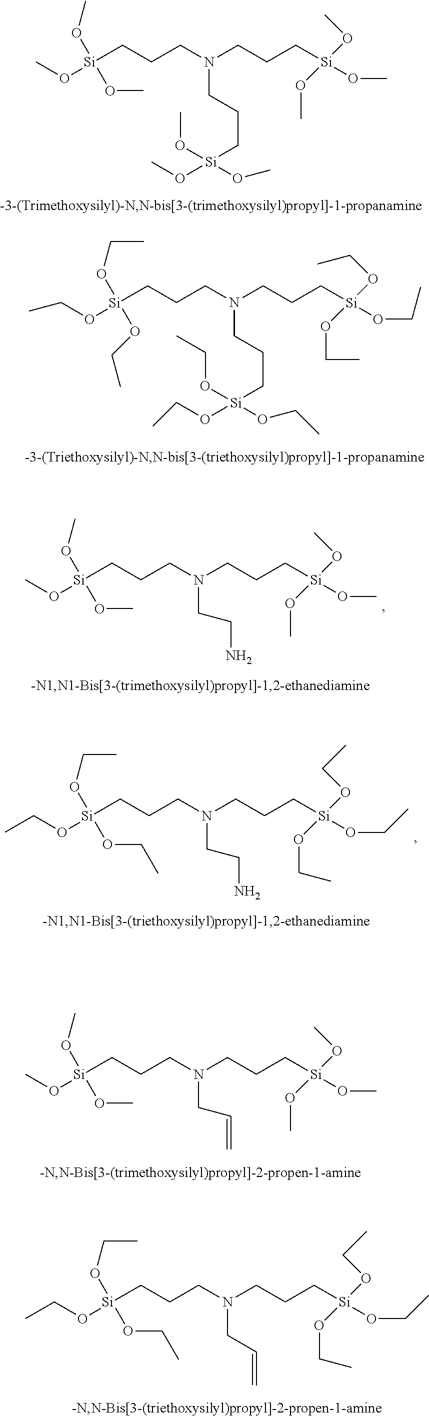

The aforementioned organic silicon compounds of formula (II) are commercially available.

Bis(trimethoxysilylpropyl)amines with the CAS number 82985-35-1 can be purchased from Sigma-Aldrich.

Bis[3-(triethoxysilyl)propyl]amines with the CAS number 13497-18-2 can be purchased from Sigma-Aldrich, for example.

N-methyl-3-(trimethoxysilyl)-N-[3-(trimethoxysilyl)propyl]-1-propanamine is alternatively referred to as bis(3-trimethoxysilylpropyl)-N-methylamine and can be purchased commercially from Sigma-Aldrich or Fluorochem.

3-(triethoxysilyl)-N,N-bis[3-(triethoxysilyl)propyl]-1-propanamine with the CAS number 18784-74-2 can be purchased for example from Fluorochem or Sigma-Aldrich.

It has also been found to be particularly advantageous when the agent for treating a keratinous material applied to the hair contains at least one organic silicon compound of formula (IV)

R₉Si(OR₁₀)ₖ(R₁₁)ₘ (IV).

The compounds of formula (IV) are organic silicon compounds selected from silanes having one, two or three silicon atoms, the organic silicon compound comprising one or more hydroxyl groups and/or hydrolysable groups per molecule.

The organic silicon compound(s) of formula (IV) may also be referred to as silanes of the alkylalkoxysilane or alkylhydroxysilane type,

R₉Si(OR₁₀)ₖ(R₁₁)ₘ (IV).

where
R₉ represents a C₁-C₁₂, alkyl group,
R₁₀ represents a hydrogen atom or a C₁-C₆ alkyl group,
R₁₁ represents a C₁-C₆ alkyl group,
k is an integer from 1 to 3, and
m stands for the integer 3-k.

In a further preferred embodiment, the agent for treating a keratin material contains, in addition to the organic silicon compounds of formula (I), at least one further organic silicon compound of formula (IV)

R₉Si(OR₁₀)ₖ(R₁₁)ₘ (IV).

where
R₉ represents a C₁-C₁₂, alkyl group,
R₁₀ represents a hydrogen atom or a C₁-C₆ alkyl group,
R₁₁ represents a C₁-C₆ alkyl group,
k is an integer from 1 to 3, and
m stands for the integer 3-k.

In a likewise preferred embodiment, the agent for treating a keratin material contains at least one further organic silicon compound of formula (IV) in addition to the organic silicon compounds of formula (II)

R₉Si(OR₁₀)ₖ(R₁₁)ₘ (IV).

where
R₉ represents a C₁-C₁₂, alkyl group,
R₁₀ represents a hydrogen atom or a C₁-C₆ alkyl group,
Ru represents a C₁-C₆ alkyl group,
k is an integer from 1 to 3, and
m stands for the integer 3-k.

In a further preferred embodiment, the agent for treating a keratin material contains, in addition to the organic silicon compounds of formula (I) and (II), at least one further organic silicon compound of formula (IV)

R₉Si(OR₁₀)ₖ(R₁₁)ₘ (IV).

where
R₉ represents a C₁-C₁₂, alkyl group,
R₁₀ represents a hydrogen atom or a C₁-C₆ alkyl group,
R₁₁ represents a C₁-C₆ alkyl group,
k is an integer from 1 to 3, and
m stands for the integer 3-k.

In the organic silicon compounds of formula (IV), the radical R₉ represents a C₁-C₁₂, alkyl group. This C₁-C₁₂, alkyl group is saturated and can be linear or branched. Preferably R₉ stands for a linear C₁-C₈ alkyl group. Preferably R₉ stands for a methyl group, an ethyl group, an n-propyl group, an n-butyl group, an n-pentyl group, an n-hexyl group, an n-octyl group or an n-dodecyl group. Particularly preferably, R₉ represents a methyl group, an ethyl group or an n-octyl group.

In the organic silicon compounds of formula (IV), the radical R₁₀ represents a hydrogen atom or a C₁-C₆ alkyl group. It is particularly preferred that R₁₀ represents a methyl group or an ethyl group.

In the organic silicon compounds of formula (IV), the radical R₁₁ represents a C₁-C₆ alkyl group. It is particularly preferred that R₁₁ represents a methyl group or an ethyl group.

Furthermore k stands for a whole number from 1 to 3, and m stands for the whole number 3-k. If k stands for the number 3, then m is equal to 0. If k stands for the number 2, then m is equal to 1. If k stands for the number 1, then m is equal to 2.

A very high “anti-pollution” effect could be obtained if the agent for treating a keratinous material contains at least one organic silicon compound of formula (IV) in which the radical k represents the number 3. In this case the rest m stands for the number 0.

Organic silicon compounds of the formula (IV) that are particularly suitable for solving the problem are

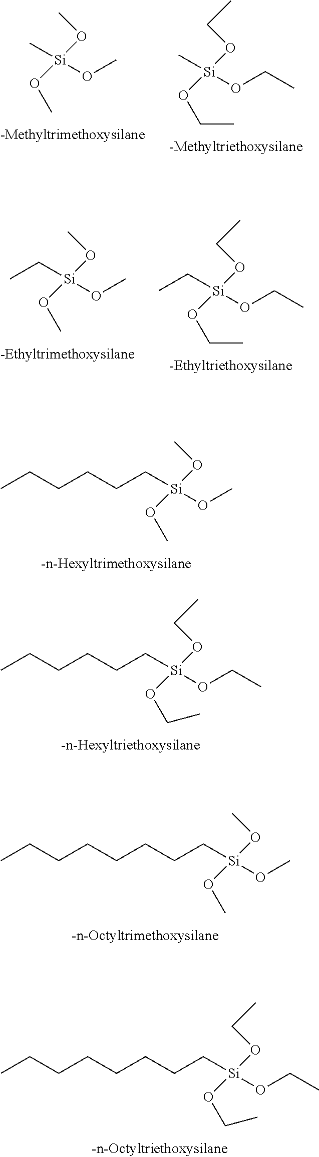

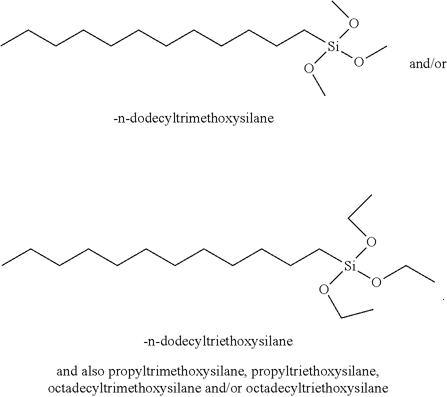

The organic silicon compounds described above are reactive compounds. In this context, it has been found to be preferred if the agent for treating a keratinous material contains—based on the total weight of the agent for treating a keratinous material—one or more organic silicon compounds in a total amount of about 0.1 to about 30% by weight, preferably from about 0.2 to about 25% by weight, particularly preferably from about 0.25 to about 20% by weight and most preferably from about 0.5 to about 15% by weight.

To achieve a particularly good “anti-pollution” effect, it is particularly advantageous to use the organic silicon compounds of formula (I) and/or (II) in certain ranges of amounts in the agent for treating a keratinous material. Particularly preferably, the agent for treating a keratinous material contains—based on the total weight of the agent for treating a keratinous material—one or more organic silicon compounds of the formula (I) and/or (II) in a total amount of about 0.1 to about 10% by weight, preferably from about 0.2 to about 5% by weight and particularly preferably from about 0.5 to about 3% by weight.

Furthermore, it has been found to be particularly preferred if the organic silicon compounds of formula (IV) are also present in the agent for treating a keratinous material in certain ranges of amounts. Particularly preferably, the agent for treating a keratinous material contains—based on the total weight of the agent for treating a keratinous material—one or more organic silicon compounds of the formula (IV) in a total amount of about 0.1 to about 20% by weight, preferably from about 2 to about 15% by weight and particularly preferably from about 4 to about 9% by weight.

In the course of the work leading to the present disclosure, it was found that particularly stable and uniform films could be obtained on the keratinous material if the agent for treating a keratinous material contains at least two organic silicon compounds which are structurally different from one another and each contain one to three silicon atoms.

In another preferred embodiment, an agent for treating a keratinous material contains two structurally different organic silicon compounds, each containing one to three silicon atoms.

In an explicitly very particularly preferred embodiment, the composition for treating a keratinous material comprises at least one organic silicone compound of formula (I) selected from the group consisting of (3-aminopropyl)triethoxysilane and (3-aminopropyl)trimethoxysilane, and additionally containing at least one organic silicone compound of formula (IV) selected from the group consisting of methyltrimethoxysilane, methyltriethoxysilane, ethyltrimethoxysilane and ethyltriethoxysilane.

In another preferred embodiment, the composition for treating a keratinous material comprises at least one organic silicone compound of formula (II) selected from the group consisting of (bis(trimethoxysilylpropyl)amine, bis[3-(trimethoxysilyl)propyl]amine and N-methyl-3-(trimethoxysilyl)-N-[3-(trimethoxysilyl)propyl]-1-propanamine, and additionally contains at least one organic silicone compound of formula (IV) selected from the group consisting of methyltrimethoxysilane, methyltriethoxysilane, ethyltrimethoxysilane and ethyltriethoxysilane.

In another preferred embodiment, an agent for treating a keratinous material comprises, based on the total weight of the cosmetic agent:
from about 0.5 to about 3 weight % of at least one first organic silicon compound selected from the group consisting of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and (2-dimethylaminoethyl)triethoxysilane, and From about 3.2 to 10 wt. % of at least one second organic silicon compound selected from the group consisting of methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilane, octadecyltrimethoxysilane and octadecyltriethoxysilane.

Even the addition of small amounts of water leads to hydrolysis in the case of organic silicon compounds having at least one hydrolyzable group. The hydrolysis products and/or organic silicon compounds having at least one hydroxyl group can react with one another in a condensation reaction. For this reason, both the organosilicon compounds having at least one hydrolyzable group and their hydrolysis and/or condensation products may be present in the composition. When organosilicon compounds having at least one hydroxyl group are used, both the organic silicon compounds having at least one hydroxyl group and their condensation products may be present in the composition.

A condensation product is understood to be a product formed by the reaction of at least two organic silicon compounds each having at least one hydroxyl group or hydrolyzable group per molecule with elimination of water and/or with elimination of an alkanol. The condensation products can be, for example, dimers, but also trimers or oligomers, with the condensation products being in equilibrium with the monomers. Depending on the amount of water used or consumed in the hydrolysis, the equilibrium of monomeric organic silicon compounds shifts to condensation product.

A second ingredient of the agent for the treatment of a keratinous material is rutin sulfate.

The term “rutin sulfate” includes mono-, di-, tri-, tetra- or polysulfates of rutin and mixtures of these rutin sulfates.

Rutin is a flavonoid and a glycoside of quercetin with the disaccharide rutinose, which is composed of rhamnose and glucose. Rutin is formed by many plants as a pigment to protect against UV radiation.

Rutin sulfate is able to scavenge free radicals and thus render them harmless. Due to its anionic charge, it binds particularly well to the film formed by the at least one organosilicon compound on the keratinous material.

A suitable rutin sulfate is available, for example, under the name RonaCare® Rutinsulfate (INCI: Disodium Rutinyl Disulfate) available from Merck KGaA.

Particularly preferably, the agent for treating a keratinous material contains—based on the total weight of the agent for treating a keratinous material-rutin sulfate in a total amount of about 0.1-10% by weight, preferably from about 0.5-about 8% by weight and particularly preferably from about 1-6% by weight.

Preferred combinations of active ingredients include:
(3-aminopropyl)trimethoxysilane and rutin sulfate,
(3-aminopropyl)triethoxysilane and rutin sulfate,
(2-aminoethyl)trimethoxysilane and rutin sulfate,
(2-aminoethyl)triethoxysilane and rutin sulfate,
(3-dimethylaminopropyl)trimethoxysilane and rutin sulfate,
(3-dimethylaminopropyl)triethoxysilane and rutin sulfate,
(2-dimethylaminoethyl)trimethoxysilane and rutin sulfate,
(2-dimethylaminoethyl)triethoxysilane and rutin sulfate,
3-(trimethoxysilyl)-N-[3-(trimethoxysilyl)propyl]-1-propanamine and rutin sulfate,
3-(triethoxysilyl)-N-[3-(triethoxysilyl)propyl]-1-propanamine and rutin sulfate,
N-methyl-3-(trimethoxysilyl)-N-[3-(trimethoxysilyl)propyl]-1-propanamine and rutin sulfate,
N-methyl-3-(triethoxysilyl)-N-[3-(triethoxysilyl)propyl]-1-propanamine and rutin sulfate,
2-[bis[3-(trimethoxysilyl)propyl]amino]ethanol and rutin sulfate,
2-[bis[3-(triethoxysilyl)propyl]amino]ethanol and rutin sulfate,
3-(trimethoxysilyl)-N,N-bis[3-(trimethoxysilyl)propyl]-1-propanamine and rutin sulfate,
3-(triethoxysilyl)-N,N-bis[3-(triethoxysilyl)propyl]-1-propanamine and rutin sulfate,
N1,N1-bis[3-(trimethoxysilyl)propyl]-1,2-ethanediamine and rutin sulfate,
N1,N1-bis[3-(triethoxysilyl)propyl]-1,2-ethanediamine and rutin sulfate,
N,N-bis[3-(trimethoxysilyl)propyl]-2-propen-1-amine and rutin sulfate,
N,N-bis[3-(triethoxysilyl)propyl]-2-propen-1-amine and rutin sulfate,
Methyl trimethoxysilane and rutin sulfate,
Methyltriethoxysilane and rutin sulfate,
Ethyl trimethoxysilane and rutin sulfate,
Ethyltriethoxysilane and rutin sulfate,
Octyltrimethoxysilane and rutin sulfate,
Octyltriethoxysilane and rutin sulfate,
Dodecyl trimethoxysilane and rutin sulfate,
Dodecyltriethoxysilane and rutin sulfate,
(3-aminopropyl)trimethoxysilane, methyltrimethoxysilane and rutin sulfate,
(3-aminopropyl)trimethoxysilane, methyltrimethoxysilane and rutin sulfate,
3-(trimethoxysilyl)-N-[3-(trimethoxysilyl)propyl]-1-propanamine, methyltrimethoxysilane and
rutin sulfate,
3-(triethoxysilyl)-N-[3-(triethoxysilyl)propyl]-1-propanamine, methyltrimethoxysilane and rutin sulfate,
N-methyl-3-(trimethoxysilyl)-N-[3-(trimethoxysilyl)propyl]-1-propanamine,
methyltrimethoxysilane and rutin sulfate,
N-methyl-3-(triethoxysilyl)-N-[3-(triethoxysilyl)propyl]-1-propanamine, methyltrimethoxysilane and rutin sulfate,
(3-aminopropyl)trimethoxysilane, methyltriethoxysilane and rutin sulfate,
(3-aminopropyl)triethoxysilane, methyltriethoxysilane and rutin sulfate,
3-(trimethoxysilyl)-N-[3-(trimethoxysilyl)propyl]-1-propanamine, methyltriethoxysilane and rutin sulfate,
3-(triethoxysilyl)-N-[3-(triethoxysilyl)propyl]-1-propanamine, methyltriethoxysilane and rutin sulfate,
N-methyl-3-(trimethoxysilyl)-N-[3-(trimethoxysilyl)propyl]-1-propanamine,
methyltriethoxysilane and rutin sulfate,
N-methyl-3-(triethoxysilyl)-N-[3-(triethoxysilyl)propyl]-1-propanamine, methyltriethoxysilane, and rutin sulfate.

In particular, the features for treating a keratinous material may comprise a feature for cleaning a keratinous material, a feature for maintaining a keratinous material, a feature for maintaining and cleaning a keratinous material, and/or a feature for temporarily reshaping a keratinous material.

Further ingredients of the hair treatment products are described below, which may be contained in the products in addition to the mandatory ingredients described above.

It may be preferred that the agent for treating a keratinous material further comprises from about 0.001 to about 20% by weight of at least one quaternary or cationized compound. This applies in particular to agents for the care of a keratinous material and agents for the care and cleaning of a keratinous material. Cationized compounds have at least one amine group, which is protonated in a correspondingly acidic environment.

It is preferred that the at least one quaternary or cationized compound is selected from at least one of the groups consisting of:
i) the monoalkylquats, and/or
ii) the esterquats, and/or
(iii) the quaternary imidazolines of formula (Tkat2),

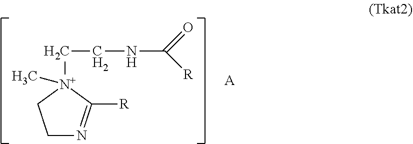

in which the radicals R independently of one another each represent a saturated or unsaturated, linear or branched hydrocarbon radical having a chain length of 8 to 30 carbon atoms and A represents a physiologically tolerated anion, and/or
iv) the cationized amidoamines, and/or
v) Poly(methacryloyloxyethyltrimethylammonium compounds), and/or;
vi) quaternized cellulose derivatives, in particular polyquaternium 10, polyquaternium-24, polyquaternium-27, polyquaternium-67, polyquaternium-72, and/or
vii) cationic alkyl polyglycosides, and/or
viii) cationized honey, and/or
ix) cationic guar derivatives, and/or
x) cationized chitosan, and/or
xi) polymeric dimethyldiallylammonium salts and copolymers thereof with esters and amides of acrylic acid and methacrylic acid, in particular polyquaternium-7, and/or
xii) copolymers of vinylpyrrolidone with quaternized derivatives of dialkylaminoalkyl acrylate and methacrylate, especially polyquaternium-11, and/or
xiii) vinylpyrrolidone-vinylimidazolium methochloride copolymers, in particular polyquaternium-16, and/or
xiv) quaternized polyvinyl alcohol, and/or
xv) polyquaternium-74,
   and mixtures thereof

It is particularly preferred that the hair treatment agent contains a monoalkyl quat, a cationized amidoamine and/or a cationic hompolymer falling under the INCI designation polyquaternium-37 as quaternary or cationized compounds.

It may be preferred that the agent for treating a keratinous material further comprises from about 0.01 to about 60% by weight of a surfactant mixture of anionic and amphoteric/zwitterionic surfactants. This applies in particular to agents for cleaning a keratinous material and agents for maintaining and cleaning a keratinous material.

Suitable anionic surfactants are preferably added to the composition for treating a keratinous material-based on the total weight of the composition—in amounts of from about 0.05 to about 45% by weight, more preferably from about 1 to about 25% by weight, particularly preferably from about 2 to about 17.5% by weight and especially from about 3 to about 15% by weight.

Suitable anionic surfactants that may be used in the composition to treat a keratinous material include:
straight-chain or branched, saturated or mono- or polyunsaturated alkyl sulfonates with 8 to 24 carbon atoms,
linear alpha-olefin sulphonates with 8 to 24 C atoms,
Alkyl sulfates and alkyl polyglycol ether sulfates of the formula R—O—(CH₂—CH₂O)n-SO₃X, in which R is preferably a straight-chain or branched, saturated or mono- or polyunsaturated alkyl or alkenyl radical containing 8 to 24 carbon atoms, n is 0 or 1 to 12 and X is an alkali metal or alkaline earth metal ion or protonated triethanolamine or the ammonium ion, and
Sulfonates of unsaturated fatty acids with 8 to 24 C-atoms and 1 to 6 double bonds,

Particularly preferred anionic surfactants are straight-chain or branched alkyl ether sulfates containing an alkyl radical with 8 to 18 and in particular with 10 to 16 carbon atoms and 1 to 6 and in particular 2 to 4 ethylene oxide units. Very preferably, the surfactant mixture of anionic and amphoteric/zwitterionic surfactants contains sodium lauryl ether sulfate (INCI: sodium laureth sulfate) and very preferably sodium lauryl ether sulfate with 2 ethylene oxide units.

Amphoteric surfactants, also known as zwitterionic surfactants, are surface-active compounds that contain at least one quaternary ammonium group and at least one —COO— or —SO₃— group in the molecule. Amphoteric/zwitterionic surfactants also include surface-active compounds which, in addition to a C₈-C₂₄ alkyl or acyl group, contain at least one free amino group and at least one —COOH or —SO₃H group and are capable of forming internal salts.

Suitable amphoteric/zwitterionic surfactants can be added in the composition for treating a keratinous material—based on the total weight of the composition—preferably in amounts of about 0.05 to about 20% by weight, more preferably of about 0.25 to about 15% by weight, particularly preferably of about 0.5 to about 10 and especially of about 1 to about 5% by weight.

Particularly suitable amphoteric/zwitterionic surfactants include those known under the INCI designation cocamidopropyl betaine and disodium cocoamphodiacetate.

The total amount of anionic and amphoteric/zwitterionic surfactants—based on the total weight of the agent for treating a keratinous material—is preferably from about 0.1 to about 40% by weight in total, more preferably from about 1 to about 35% by weight and further preferably from about 3 to about 30% by weight.

It may be preferred that the agent for treating a keratinous material further comprises a strengthening compound, preferably selected from the group consisting of waxes, synthetic polymers, and mixtures thereof.

In order to meet the different requirements for agents for the treatment of a keratinous material in the form of an agent for the temporary reshaping of a keratinous material (=styling agent), a large number of synthetic polymers have already been developed as strengthening compounds which can be used in an agent for the treatment of a keratinous material. Alternatively, or additionally, waxes are used as strengthening compounds. Ideally, the polymers and/or waxes, when applied to the keratinous material, result in a polymer film or sheet that, on the one hand, gives the hairstyle a strong hold, but, on the other hand, is sufficiently flexible not to break when stressed.

The synthetic polymers can be divided into cationic, anionic, nonionic and amphoteric strengthening polymers.

Suitable synthetic polymers include, for example, polymers with the following INCI names: Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates Copolymer, Acrylates/C₁-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/Hydroxyesters Acrylates Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate and Styrene/VP Copolymer. Cellulose ethers such as hydroxypropyl cellulose, hydroxyethyl cellulose, and methyl hydroxypropyl cellulose are also suitable.

Homopolyacrylic acid (INCI: Carbomer), which is commercially available under the name Carbopol® in various forms, is suitable as a firming compound.

Preferably, the fixing compound a polymer containing vinylpyrrolidone. Particularly preferably, the firming compound comprises a polymer selected from the group consisting of polyvinylpyrrolidone (PVP), vinylpyrrolidone-vinyl acetate copolymer (VP/VA copolymer), vinyl caprolactam/VP/dimethylaminoethyl methacrylate copolymer (INCI), VP/DMAPA acrylates copolymer (INCI), and mixtures thereof.

Another preferred solidifying compound is octylacrylamide/acrylates/butylaminoethyl methacrylate copolymer (INCI), which is marketed under the name “Amphomer®” by Akzo Nobel.

Accordingly, it is particularly preferred that the firming compound comprises a synthetic polymer selected from the group consisting of polyvinylpyrrolidone (PVP), vinylpyrrolidone-vinyl acetate copolymer (VP/VA copolymer), vinyl caprolactam/VP/dimethylaminoethyl methacrylate copolymer (INCI), VP/DMAPA acrylates copolymer (INCI), octylacrylamide/acrylates/butylaminoethyl methacrylate copolymer (INCI), and mixtures thereof.

The cosmetic compound may contain, in addition or as an alternative to a synthetic polymer, at least one natural or synthetic wax having a melting point above 37° C. as a firming compound.

Natural or synthetic waxes may be solid kerosenes or isoparaffins, vegetable waxes such as candelilla wax, carnauba wax, esparto grass wax, Japan wax, cork wax, sugar cane wax, ouricury wax, montan wax, sunflower wax, fruit waxes and animal waxes such as beeswaxes and other insect waxes, Whale wax, shellac wax, wool wax and brushing grease, furthermore mineral waxes, such as ceresin and ozokerite or petrochemical waxes, such as petrolatum, kerosene waxes, microwaxes of polyethylene or polypropylene and polyethylene glycol waxes can be used. It may be advantageous to use hydrogenated or cured waxes. Chemically modified waxes, in particular hard waxes such as montan ester waxes, sasol waxes, and hydrogenated jojoba waxes, can also be used.

Also suitable are triglycerides of saturated and optionally hydroxylated C16-30 fatty acids, such as hydrogenated triglyceride fats (hydrogenated palm oil, hydrogenated coconut oil, hydrogenated castor oil), glyceryl tribehenate or glyceryl tri-12-hydroxystearate, furthermore synthetic full esters of fatty acids and glycols (for example Syncrowax®) or polyols with 2 to 6 C atoms, fatty acid monoalkanolamides with a C12-22 acyl radical and a C2-4 alkanol radical, esters of saturated and/or unsaturated, branched and/or unbranched alkanecarboxylic acids with a chain length of 1 to 80 C atoms and saturated and/or unsaturated branched and/or unbranched alcohols of a chain length of 1 to 80 C atoms, including, for example, synthetic fatty acid fatty alcohol esters such as stearyl stearate or cetyl palmitate, esters of aromatic carboxylic acids, dicarboxylic acids or hydroxycarboxylic acids (for example, 12-hydroxystearic acid) and saturated and/or unsaturated branched and/or unbranched alcohols having a chain length of 1 to 80 carbon atoms, lactides of long-chain hydroxycarboxylic acids and full esters of fatty alcohols and di- and tricarboxylic acids, for example dicetyl succinate or dicetyl/stearyl adipate, and mixtures of these substances.

The wax components can also be selected from the group of esters of saturated, unbranched alkanecarboxylic acids having a chain length of 14 to 44 carbon atoms and saturated, unbranched alcohols having a chain length of 14 to 44 carbon atoms, provided that the wax component or the totality of wax components are solid at room temperature. The wax components can be selected, for example, from the group of C16-36 alkyl stearates, C10-40 alkyl stearates, C2-40 alkyl isostearates, C20-40 dialkyl esters of dimer acids, C18-38 alkyl hydroxystearoyl stearates, C20-40 alkyl erucates, and C30-50 alkyl beeswax and cetearyl behenate can also be used. Silicone waxes, for example stearyltrimethylsilane/stearyl alcohol, may also be beneficial. Preferred wax components are the esters of saturated monohydric C20-C60 alcohols and saturated C8-C30 monocarboxylic acids, in particular a C20-C40 alkyl stearate preferably available under the name Kesterwachs® K82H from Koster Keunen Inc.

Natural, chemically modified and synthetic waxes can be used alone or in combination. Thus, several waxes can also be used. Furthermore, a number of wax compounds, possibly mixed with other additives, are also commercially available. The products sold under the designations “Special Wax 7686 OE” (a mixture of cetyl palmitate, beeswax, microcrystalline wax and polyethylene with a melting range of about 73-75° C.; manufacturer: Kahl & Co), Polywax® GP 200 (a mixture of stearyl alcohol and polyethylene glycol stearate with a melting point of about 47-51° C.; manufacturer: Croda) and “Softceresin® FL 400” (a vaseline/vaseline oil/wax mixture with a melting point of about 50-54° C.; manufacturer: Parafluid Mineral Oil Company) are examples of mixtures that can be used.

Preferably, the wax is selected from carnauba wax (INCI: Copernicia Cerifera Cera) Beeswax (INCI: Beeswax), petrolatum (INCI), microcrystalline wax and especially mixtures thereof.

Preferred blends include the combination of carnauba wax (INCI: Copernicia Cerifera Cera), petrolatum and microcrystalline wax or the combination of beeswax (INCI: Beeswax) and Petrolatum.

Wax or the wax components should be solid at 25° C. and should melt in the range>37° C.

The composition for treating a keratinous material contains a firming compound preferably in a total amount of about 0.5 to about 50% by weight, preferably from about 1 to about 40% by weight, more preferably from about 1.5 to about 30% by weight, even more preferably from about 2 to about 25% by weight, based on the total weight of the cosmetic composition.

Other suitable ingredients include nonionic surfactants, nonionic polymers, anionic polymers, (other) cationic polymers, fatty substances, waxes, protein hydrolysates, amino acids, oligopetides, vitamins, provitamins, vitamin precursors, betaines, bioquinones, purine (derivatives), taurine (derivatives), conditioning agents, plant extracts, silicones, ester oils, UV light filters, structuring agents, thickening agents, electrolytes, pH-adjusting agents, swelling agents, colorants, anti-dandruff agents, complexing agents, opacifiers, pearlescent agents, pigments, stabilizing agents, propellants, antioxidants, perfume oils, and/or preservatives.

In preferred embodiments 1 to 48, the preferred organic silicon compounds containing one to three silicon atoms are combined with the most preferred rutin sulfate.

| | Silane compound | other ingredients |
|---|---|---|
| 1 | (3-Aminopropyl)trimethoxysilane | Disodium Rutinyl |
| | | Disulfate (INCI) |
| 2 | (3-Aminopropyl)triethoxysilan | Disodium Rutinyl |
| | | Disulfate (INCI) |
| 3 | (2-Aminoethyl)trimethoxysilane | Disodium Rutinyl |
| | | Disulfate (INCI) |
| 4 | (2-Aminoethyl)triethoxysilane | Disodium Rutinyl |
| | | Disulfate (INCI) |
| 5 | (3-Dimethylaminopropyl)trimethoxysilane | Disodium Rutinyl |
| | | Disulfate (INCI) |
| 6 | (3-Dimethylaminopropyl)triethoxysilane | Disodium Rutinyl |
| | | Disulfate (INCI) |
| 7 | (2-Dimethylaminoethyl)trimethoxysilane | Disodium Rutinyl |
| | | Disulfate (INCI) |
| 8 | (2-Dimethylaminoethyl)triethoxysilane | Disodium Rutinyl |
| | | Disulfate (INCI) |
| 9 | 3-(trimethoxysilyl)-N-[3- | Disodium Rutinyl |
| | (trimethoxysilyl)propyl]-1-propanamine | Disulfate (INCI) |
| 10 | 3-(triethoxysilyl)-N-[3- | Disodium Rutinyl |
| | (triethoxysilyl)propyl]-1-propanamine | Disulfate (INCI) |
| 11 | N-methyl-3-(trimethoxysilyl)-N-[3- | Disodium Rutinyl |
| | (trimethoxysilyl)propyl]-1-propanamine | Disulfate (INCI) |
| 12 | N-Methyl-3-(triethoxysilyl)-N-[3- | Disodium Rutinyl |
| | (triethoxysilyl)propyl]-1-propanamine | Disulfate (INCI) |
| 13 | 2-[Bis[3-(trimethoxysilyl) | Disodium Rutinyl |
| | propyl]amino]-ethanol | Disulfate (INCI) |
| 14 | 2-[bis[3-(triethoxysilyl) | Disodium Rutinyl |
| | propyl]amino]ethanol | Disulfate (INCI) |
| 15 | 3-(trimethoxysilyl)-N,N-bis[3- | Disodium Rutinyl |
| | (trimethoxysilyepropyl]-1-propanamine | Disulfate (INCI) |
| 16 | 3-(triethoxysilyl)-N,N-bis[3- | Disodium Rutinyl |
| | (triethoxysilyl)propyl]-1-propanamine | Disulfate (INCI) |
| 17 | N1,N1-Bis[3-(trimethoxysilyl) | Disodium Rutinyl |
| | propyl]-1,2-ethanediamine | Disulfate (INCI) |
| 18 | N1,N1-Bis[3-(triethoxysilyl) | Disodium Rutinyl |
| | propyl]-1,2-ethanediamine | Disulfate (INCI) |
| 19 | N,N-Bis[3-(trimethoxysilyl) | Disodium Rutinyl |
| | propyl]-2-propen-1-amine | Disulfate (INCI) |
| 20 | N,N-Bis[3-(triethoxysilyl) | Disodium Rutinyl |
| | propyl]-2-propen-1-amine | Disulfate (INCI) |
| 21 | Methyltrimethoxysilane | Disodium Rutinyl |
| | | Disulfate (INCI) |
| 22 | Methyltriethoxysilane | Disodium Rutinyl |
| | | Disulfate (INCI) |
| 23 | Ethyltrimethoxysilane | Disodium Rutinyl |
| | | Disulfate (INCI) |
| 24 | Ethyltriethoxysilane | Disodium Rutinyl |
| | | Disulfate (INCI) |
| 25 | Octyltrimethoxysilane | Disodium Rutinyl |
| | | Disulfate (INCI) |
| 26 | Octyltriethoxysilane | Disodium Rutinyl |
| | | Disulfate (INCI) |
| 27 | Dodecyltrimethoxysilane | Disodium Rutinyl |
| | | Disulfate (INCI) |
| 28 | Dodecyltriethoxysilane | Disodium Rutinyl |
| | | Disulfate (INCI) |
| 29 | Propyltrimethoxysilane | Disodium Rutinyl |
| | | Disulfate (INCI) |
| 30 | Propyltriethoxysilane | Disodium Rutinyl |
| | | Disulfate (INCI) |
| 31 | Hexyltrimethoxysilane | Disodium Rutinyl |
| | | Disulfate (INCI) |
| 32 | Hexyltriethoxysilane | Disodium Rutinyl |
| | | Disulfate (INCI) |
| 33 | Octadecyltriethoxysilane | Disodium Rutinyl |
| | | Disulfate (INCI) |
| 34 | Octadecyltriethoxysilane | Disodium Rutinyl |
| | | Disulfate (INCI) |
| 35 | (3-Aminopropyl)triethoxysilan + | Disodium Rutinyl |
| | Methyltrimethoxysilan | Disulfate (INCI) |
| 36 | (3-Aminopropyl)triethoxysilane + | Disodium Rutinyl |
| | Methyltriethoxysilane | Disulfate (INCI) |
| 37 | (3-Aminopropyl)triethoxysilane + | Disodium Rutinyl |
| | Ethyltrimethoxysilane | Disulfate (INCI) |
| 38 | (3-Aminopropyl)triethoxysilane + | Disodium Rutinyl |
| | Ethyltriethoxysilane | Disulfate (INCI) |
| 39 | (3-Aminopropyl)triethoxysilane + | Disodium Rutinyl |
| | Propyltrimethoxysilane | Disulfate (INCI) |
| 40 | (3-Aminopropyl)triethoxysilane + | Disodium Rutinyl |
| | Propyltriethoxysilane | Disulfate (INCI) |
| 41 | (3-Aminopropyl)triethoxysilane + | Disodium Rutinyl |
| | Hexyltrimethoxysilane | Disulfate (INCI) |
| 42 | (3-Aminopropyl)triethoxysilane + | Disodium Rutinyl |
| | Hexyltriethoxysilane | Disulfate (INCI) |
| 43 | (3-Aminopropyl)triethoxysilane + | Disodium Rutinyl |
| | Octyltrimethoxysilane | Disulfate (INCI) |
| 44 | (3-Aminopropyl)triethoxysilane + | Disodium Rutinyl |
| | Octyltriethoxysilane | Disulfate (INCI) |
| 45 | (3-Aminopropyl)triethoxysilane + | Disodium Rutinyl |
| | Dodecyltrimethoxysilane | Disulfate (INCI) |
| 46 | (3-Aminopropyl)triethoxysilane + | Disodium Rutinyl |
| | Dodecyltriethoxysilane | Disulfate (INCI) |
| 47 | (3-Aminopropyl)triethoxysilane + | Disodium Rutinyl |
| | octyldecyltrimethoxysilane | Disulfate (INCI) |
| 48 | (3-Aminopropyl)triethoxysilane + | Disodium Rutinyl |
| | Octyldecyltriethoxysilane | Disulfate (INCI) |

The active ingredient combination of at least one organic silicon compound containing one to three silicon atoms and rutin sulfate may already be present in the composition for treating a keratinous material. In this embodiment, the agent for treating a keratinous material is already distributed in a ready-to-use form. In order to provide a formulation that is as stable as possible during storage, the agent itself is preferably packaged with low or no water.

Alternatively, the at least one organic silicon compound is added at most about 12 hours, preferably at most about 6 hours, more preferably at most about 1 hour, even more preferably at most about 30 minutes, and most preferably at most about 20 minutes, before application of the composition for treating a keratinous material to a base comprising all ingredients of the composition for treating a keratinous material except the at least one organic silicon compound.

For example, the user may first mix or shake an agent (α) comprising the organic silicon compound(s) with an agent (β) comprising the remaining ingredients of the agent for treating a keratinous material. The user can now apply this mixture of (α) and (β)—either directly after its preparation or after a short reaction time of about 10 seconds to about 20 minutes—to the keratinous materials. The agent (β) may contain water, in particular water in an amount >30% by weight, based on the total weight of the agent for treating keratinous materials.

Another subject matter of the present application is the use of a cosmetic agent as contemplated herein for treating a keratinous material

to reduce and/or prevent harmful effects of air and water contaminants on keratinous material, for reducing and/or preventing the formation of free radicals by air and water contaminants on a keratinous material, and/or
to render harmless free radicals formed on a keratinous material by air and water impurities.

With regard to further preferred embodiments of use, the same applies mutatis mutandis as to the cosmetic agents.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic agent for the treatment of a keratinous material, comprising
a) at least one organic silicon compound comprising one to three silicon atoms, and
b) Rutin sulfate.

2. A cosmetic agent for treating a keratinous material according to claim 1, wherein then at least one organic silicon compound comprises a silane having one, two, or three silicon atoms, wherein the organic silicon compound comprises one or more hydroxyl groups or hydrolyzable groups per molecule.

3. A cosmetic agent for treating a keratinous material according to claim 1,
wherein the at least one organic silicon compound comprises a compound of formula (I) and/or (II),
wherein in the organic silicon compound of formula (I)
R₁R₂N-L-Si(OR₃)ₐ(R₄)_{b} (I),
R₁, R₂ independently represent a hydrogen atom or a C₁-C₆ alkyl group,
L is a linear or branched C₁-C₂₀ divalent alkylene group,
R₁, R₄ independently of one another represent a C₁-C₆ alkyl group,
a, stands for an integer from 1 to 3, and
b stands for the integer 3-a, and
wherein in the organic silicon compound of formula (II)
(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),
R₅, R_{5′}, R_{5″}, R₆, R_{6′} and R_{6″} stand independently for a C₁-C₆ alkyl group,
A, A₁, A₂, A₃ and A₄ independently represent a linear or branched divalent C₁-C₂₀ alkylene group, and
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group, or a group of formula (III)
-(A″″)-Si(R₆″)_{d}″(OR₅″)_{c}″ (III),
where
c stands for an integer from 1 to 3,
d stands for the integer 3-c′,
c′ stands for an integer from 1 to 3,
d′ stands for the integer 3-c″,
c″ stands for an integer from 1 to 3,
d″ stands for the integer 3-c″,
e stands for 0 or 1,
f stands for 0 or 1,
g stands for 0 or 1, and
h stands for 0 or 1,
with the proviso that at least one of the components of e, f, g and h is different from 0.

4. Cosmetic agent for treating a keratinous material according to claim 1, wherein the agent for treating a keratinous material comprises at least one organic silicon compound of formula (I),
R₁R₂N-L-Si(OR₃)ₐ(R₄)_{b} (I),
where
R₁, R₂ both represent a hydrogen atom,
L represents a linear, divalent C₁-C₆-alkylene group,
R₃, R₄ independently represent a methyl group or an ethyl group,
a stands for the number 3, and
b stands for the number 0.

5. Cosmetic agent for treating a keratinous material according to claim 1, wherein the agent for treating a keratinous material comprises at least one organic silicon compound of formula (I) chosen from the group consisting of:
(3-Aminopropyl)trimethoxysilan,
(3-Aminopropyl)triethoxysilan,
(2-Aminoethyl)trimethoxysilan,
(2-Aminoethyl)triethoxysilan,
(3-Dimethylaminopropyl)trimethoxysilan,
(3-Dimethylaminopropyl)triethoxysilan,
(2-dimethylaminoethyl)trimethoxysilane, and
(2-Dimethylaminoethyl)triethoxysilan.

6. Cosmetic agent for treating a keratinous material according to claim 1, wherein the agent for treating a keratinous material comprises at least one organic silicon compound of formula (II), chosen from the group consisting of:
3-(trimethoxysilyl)-N-[3-(trimethoxysilyl)propyl]-1-propanamine,
3-(Triethoxysilyl)-N-[3-(triethoxysilyl)propyl]-1-propanamine,
N-methyl-3-(trimethoxysilyl)-N-[3-(trimethoxysilyl)propyl]-1-propanamine,
N-Methyl-3-(triethoxysilyl)-N-[3-(triethoxysilyl)propyl]-1-propanamine,
2-[Bis[3-(trimethoxysilyl)propyl]amino]-ethanol,
2-[bis[3-(triethoxysilyl)propyl]amino]ethanol,
3-(Trimethoxysilyl)-N,N-bis[3-(trimethoxysilyl)propyl]-1-propanamine,
3-(Triethoxysilyl)-N,N-bis[3-(triethoxysilyl)propyl]-1-propanamine,
N1,N1-bis[3-(trimethoxysilyl)propyl]-1,2-ethanediamine,
N1,N1-bis[3-(triethoxysilyl)propyl]-1,2-ethanediamine,
N,N-bis[3-(trimethoxysilyl)propyl]-2-propen-1-amine, and
N,N-bis[3-(triethoxysilyl)propyl]-2-propen-1-amine.

7. Cosmetic agent for treating a keratinous material according to claim 1, wherein the agent for treating a keratinous material comprises at least one organic silicon compound of formula (I),
R₉Si(OR₁₀)ₖ(R₁₁)ₘ (IV)
where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3-k.

8. Cosmetic agent for treating a keratinous material according to claim 1, wherein the agent for treating a keratinous material comprises at least one organic silicon compound of formula (I), chosen from the group consisting of;
Methyltrimethoxysilane,
Methyltriethoxysilane,
Ethyltrimethoxysilane,
Ethyltriethoxysilane,
Propyltrimethoxysilane,
Propyltriethoxysilane,
Hexyltrimethoxysilane,
Hexyltriethoxysilane,
Octyltrimethoxysilane,
Octyltriethoxysilane,
Dodecyltrimethoxysilane,
Dodecyltriethoxysilane,
Octadecyltrimethoxysilane, and
Octadecyltriethoxysilane

9. A cosmetic composition for treating a keratinous material according to claim 1, wherein the composition for treating a keratinous material comprises at least two structurally different organic silicon compounds, each comprising one to three silicon atoms.

10. Use of a cosmetic agent for treating a keratinous material according to claim 1.
to reduce and/or prevent harmful effects of air and water contaminants on keratinous material,
for reducing and/or preventing the formation of free radicals by air and water contaminants on a keratinous material, and/or
for rendering harmless free radicals formed on a keratinous material by air and water impurities.
